(19) United States
(12) Patent Application Publication
(10) Pub. No.: US 2004/0076989 A1
(43) Pub. Date: Apr. 22, 2004

(54) Synaptic transmission assay

(76) Inventors: Alan Fine, London (GB); Tim Bliss, London (GB); Christopher Clive Gabriel Hentschel, Geneva (CH)

Correspondence Address: PALMER & DODGE, LLP, KATHLEEN M. WILLIAMS, 111 HUNTINGTON AVENUE, BOSTON, MA, 02199, US

(21) Appl. No.: 10/465,113

(22) Filed: Jun. 19, 2003

Related U.S. Application Data
(63) Continuation of application No. PCT/GB01/05756, filed on Dec. 21, 2001.

(30) Foreign Application Priority Data
Dec. 22, 2000 (GB) 0031546.5
Jan. 30, 2001 (GB) 0102338.1

(51) Int. Cl. C12Q 1/68; G01N 33/53; G01N 33/567

(52) U.S. Cl. 435/6; 435/7.2

(57) ABSTRACT

The invention relates to a method for assessing the ability of a test compound or mixture of test compounds to modulate LTP induction by measuring the modulation of immediate early gene expression in response to the compound or mixture of compounds.

### FIELD OF THE INVENTION

The present invention relates to a method for assaying the ability of a test compound to modulate the induction or expression of long-term potentiation (LTP) of synaptic transmission in a neural system. In particular, the invention relates to a method for assaying the ability of a test compound or compounds to induce or modulate immediate-early or other reporter gene transcription and/or translation and for correlating transcription and/or translation of an immediate-early gene or a reporter gene to LTP induction.

### BACKGROUND TO THE INVENTION

There are many neurological conditions for which synaptic plasticity-enhancing therapeutic treatments, such as methods to enhance memory or to treat memory dysfunction, are under investigation. For example, memory dysfunction is associated with the aging process, as well as to neurodegenerative diseases such as Alzheimer's disease, multi-infarct dementia, head trauma and a variety of other conditions. Many compounds and treatments have been investigated in an effort to enhance cognitive processes in these and other contexts.

Learning and memory are widely regarded as being associated with synaptic changes in the brain, and in particular with long-term potentiation (LTP) of synaptic activity. LTP manifests itself as a persistent increase in the efficiency of synaptic transmission, and can be observed throughout the hippocampus as well as in many other regions of the brain.

LTP in the hippocampus is the prevailing model for memory in the mammalian brain (see Bliss and Collingridge, (1993) Nature 361:31-39) and as such has been extensively studied in researching pathological conditions which affect memory. Memory disorders are major public health problems that can have devastating effects on quality of life. As yet, there are no effective therapies for such disorders.

This is due, in large measure, to the lack of an efficient model to identify potential treatments. Potential drugs are tested by behavioural studies, or by electrophysiological studies which seek to measure LTP in situ in laboratory animals or in brain tissue slices. Behavioural studies are laborious, and expensive. Electrophysiological studies are highly time-consuming and require highly skilled personnel.

LTP is known to be associated with transcriptional activity involving de novo synthesis of mRNA encoding a variety of regulatory molecules (see below and Thomas & Hunt (1996) in Cortical Plasticity, Fazeli and Collingridge (Eds.), BIOS Scientific Publishers, Oxford, UK). The regulatory molecules whose expression is linked with LTP include zinc finger transcription factors, leucine zipper polypeptides, protein kinases and phosphatases, glutamate receptors, growth factors, proteases and cell adhesion molecules. Most of these molecules are present in a wide variety of tissues of the body and have highly diverse functions.

An assay system which is capable of assessing the ability of a test pharmaceutical to modulate or induce LTP would be highly desirable, particularly if configured to provide a high-throughput readout. Such an assay system could be employed to screen large compound libraries for lead compounds which may then be further developed in order to assess their potential as pharmaceuticals. Presently, no satisfactory high-throughput system for the identification of lead compounds for the treatment of memory disorders exists.

### SUMMARY OF THE INVENTION

It has been determined that the regulation of the expression of genes associated with LTP may be exploited to assess LTP induction, despite the ubiquitous nature of the gene products expressed by such genes.

In a first aspect, therefore, the present invention provides a method for determining whether one or more compounds is a potential modulator of long-term potentiation (LTP) in the brain, comprising the steps of:

a) providing a cell which expresses a gene under the control of a regulatory sequence naturally associated with a gene whose expression is associated with LTP;

b) contacting the cell with one or more compounds;

c) determining the ability of the compound or compounds to modulate the expression of the gene; and

d) correlating the modulation of gene expression with the ability to modulate LTP in the brain.

The present invention provides an assay methodology, which can be applied to high-throughput assays, and which exploits the gene transcription events which are associated with LTP. Although many of the genes: whose modulation of transcription is associated with LTP encode commonplace or ubiquitous polypeptides, it has surprisingly been found that monitoring their transcriptional activity can provide a useful indication of the ability of a test compound to induce or modulate LTP in the brain in vivo.

A “reporter” gene, therefore, is a gene whose expression is modulated as a result of, as a precursor to or otherwise in association with the induction of LTP in the animal brain. Preferably, the brain is a mammalian brain. A number of such genes are known in the art and are identified herein. Further genes are described below, and in Thomas & Hunt (1996) in Cortical Plasticity, Fazeli and Collingridge (Eds.), BIOS Scientific Publishers, Oxford, UK.

“Modulation of expression” is either an increase or a decrease in either the activity of the gene product encoded by the gene or the level of transcription of primary RNA transcript encoded by the gene. In the case of activity of the gene products it will be apparent to those skilled in the art that this can be affected by increasing or decreasing the levels of transcription and/or translation of the primary RNA gene product, post-translational processing of the gene product or otherwise. Advantageously, it is a result of a change in the transcriptional activity of the gene.

Preferably, modulation refers in an increase or decrease of about 20%, 50%, 100%, 250% or advantageously 500% or more in the activity of the gene product or the transcription level of the RNA in question.

The assay may be configured to operate in any cellular system which expresses a gene under the control of a regulatory sequence which is normally associated with LTP induction or expression, often via an immediate-early gene as discussed above. “Cell” or “cellular system”, as used herein, preferably refers to essentially complete cells, which are preferably animal cells, advantageously mammalian cells, and most advantageously human cells. The term can refer to artificial systems capable of replicating the transcriptional environment of a natural cell; such systems should also be configured to replicate the responses of natural cells to LTP-inducing stimuli. The term also describes cells included in tissues or tissue slices derived from animals, typically from animal brains, and to cells included in whole animals, chimeric or otherwise, such as transgenic mice or rat.

“Expression” is to be understood to refer to transcription of a coding sequence, to produce at least an RNA product. Optionally, the term is also used to describe translation of the RNA product to produce a polypeptide product. However, the invention also includes the possibility that the RNA product should itself be the final product, for example in the form of a ribozyme or other detectable RNA species.

The term “gene” is used herein in the broad sense and serves to denote that the nucleic acid can be transcribed to form a gene product. The “gene” may comprise all or some of the regulatory sequences required for transcription, or not, as the context requires; the term may refer purely to a coding sequence encoding the reporter gene product.

The gene may be a native or heterologous reporter gene. Thus, in one embodiment it is envisaged that expression of native immediate early genes may be assessed directly, for example by in situ hybridisation of nucleic acids, by immunological detection including in situ immunofluorescence, by Western blotting, by Northern analysis, S1 mapping and any other technique capable of assessing the amounts of nucleic acid or protein present in a cell or a sample.

Advantageously, expression of native immediate-early gene products is assessed by immunofluorescence. For example, expression of the genes encoding zif268 or arc proteins may be assessed using anti-zif268 or anti-arc antibodies.

The gene may also be a reporter gene comprising a heterologous coding sequence. The reporter gene itself may be any gene whose gene product is detectable or participates in a reaction with a detectable end-point For example, therefore, the gene product may be an RNA product, such as a ribozyme or an antisense RNA molecule, which is capable of participating in an enzymatic reaction or of modulating the expression of a further gene. Preferably, however, the gene product is a polypeptide gene product, which is advantageously detectable by optical means or catalysis a reaction which has a chromogenic end-point. For example, the gene product is a fluorescent polypeptide, such as green fluorescent protein (GFP) or a variant thereof, including cyan fluorescent protein and other engineered forms of GFP, red fluorescent protein, or a polypeptide capable of inducing luminescence, such as a luciferase. Moreover, the gene product may be an enzyme, such as β-lactamase, β-glucoronidase, neomycin phosphotransferase, alkaline phosphatase, guanine xanthine phosphoribosyl-transferase or β-galactosidase, or a peptide sequence specifically bound by a particular coloured, fluorescent, or otherwise detectable molecule.

“Detectable”, as used herein, means that expression of the gene in question either is or gives rise to an event which can be measured, either quantitatively or qualitatively, or both, and which can be correlated to the modulation of the expression of the reporter gene. The expression may be positively or negatively regulated, that is may be enhanced or reduced.

Optical detection of gene product expression, whether performed directly or indirectly, is desirable as it allows the assay according to the invention to be automated in a straight-forward manner. Methods for optical detection are well known in the art and are coupled with high throughput screening strategies in order to permit screening of large numbers of compounds in cell-based assays. In general, cells may be plated out in monolayer cultures and contacted with test compounds in accordance with the present invention under conventional conditions, for instance as set out below. The generation of a luminescent, fluorescent or other optical signal may then be monitored in the cells using automated optical readers, as are known in the art, in order to assess reporter gene activation in response to compound addition. Results may then be collated manually or, preferably, using a computer-based system, in order to identify those compounds which are capable of inducing reporter gene expression in the cells.

Expression of the reporter gene is under the control of a regulatory sequence typically associated with an immediate-early gene as discussed above. This means that at least the transcription and/or translation of the reporter gene is capable of being modulated in response to stimuli which induce or modulate LTP or which are otherwise associated with LTP. The regulatory sequence may be a promoter, an enhancer or a combination of both; advantageously, it is an enhancer element. Promoters and enhancers are known in the art and may; be: derived from any immediate-early gene associated with LPT. Preferably, the promoter and/or enhancer is derived from a gene selected from the group consisting of zif268 (also known as NGFI-A, krox24, egr1; Milbrandt, (1987) Science 238, 797-799), arc (also known as arg 3.1 (Link, W. et al., Proc. Natl. Acad Sci. USA 92, 5734-5738 (1995); Lyford, G. L. et al., Neuron 14, 433-445 (1995), egr3 (Yamagata et al., (1994) Learn. Mem. 1:140-152), CREB (Gallin & Geenberg, (1995) Curr. Opin. Neurobiol. 5:365-374), c-fos, fra-1, fra-2, fosB, c-jun, junB and junD (see Dragunow et al, (1989) Neurosci. Lett. 101:274-280; Jeffery et al., (1990) Mol. Brain. Res. 8:267-274; Nickolaev et al., (1991) Brain. Res. 560:346-349; and Demmer et al., (1993) Mol. Brain. Res. 17:279-286), C/EPB (Albertim et al., (1994) Cell 76:1099-1114), CAMKII (Roberts et al., (1995) Br. J. Pharmacol. 116:SSpP348), PKC, PKA, homer, frequenin, BDNF, AKAP150, ERK-2, Raf-B, BAD-2, and NMDA-, AMPA- and metabotropic-glutamate receptors (see, in general, the references cited in Thomas & Hunt (1996) in Cortical Plasticity, Fazeli and Collingridge (Eds.), BIOS Scientific Publishers, Oxford, UK).

Preferably, the regulatory sequence is derived from a zif268 gene or any gene regulated by cAMP via a cAMP response element (CRE). For example, therefore, the regulatory element is a zif268 promoter or comprises one or more CREs.

In an alternative embodiment, the reporter gene may be controlled by a regulatory sequence which is susceptible to modulation by an immediate-early gene product whose expression is associated with LTP. For example, therefore, a reporter gene is placed under the control of a promoter which is transactivatable by the transcription factor zif268 or by arc. LTP-associated induction of zif268/arc then leads to a concomitant upregulation of reporter gene expression. Similarly, the expression of any of the above genes in native form may be detected directly using suitable techniques. Further downstream processed associated with LTP induction may also be detected optically, such as increased rates of presynaptic neurotransmitter release.

### BRIEF DESCRIPTION OF THE FIGURES

FIG. 1. Gross hippocampal anatomy is normal in zif268−/− mice. (a) NeuN immunoreactivity, (b) parvalbumin staining and (c) synaptophysin imnmunoreactivity all appeared similar in −/− (right hand column) and +/+ (left hand column) mice.

FIG. 2. Short-term synaptic plasticity and LTP in the dentate gyrus of anaesthetised zif268 mutant mice. (a) At low stimulus intensities, paired-pulse facilitation of EPSP amplitude shows the same dependence on inter-stimulus interval (ISI) in −/− (filled circles, n=5) and +/+ mice (open circles, n 4). Sample responses show facilitation at 15 ms ISI in a −/− mouse. (b) At stimulus intensities just above threshold for evoking a population spike, paired-pulse depression and facilitation of the population spike also show the same dependence, on ISI in −/− and +/+ mice. Sample responses show depression (10 ms ISI) and facilitation.(50 mrs ISI) from a −/− mouse. (c, d) LTP of the population spike (c) and the field EPSP (d) in −/− and +/+ mice (filled circles, n=5, and open circles, n=4, respectively). Mean changes in the amplitude of the population spike are plotted in (c), and percentage changes in the slope of the field EPSP in (d), both with respect to the mean values in the 10 min preceding tetanic stimulation (arrows). Test stimuli were given to the perforant path at a frequency of 1 per 30 s. A similar level of potentiation of the population spike and EPSP was seen in +/+ and mice −/− for the first hour post-tetanus.

FIG. 3. LTP in the dentate gyrus of awake zif268 mutant mice. (a) Stimulus intensity required to evoke field potentials with comparable EPSP slopes and population spike amplitudes was independent of genotype. Sample responses are shown for mice of each genotype before (dotted line) and 10 min after (solid line) tetanic stimulation. Values for the mean amplitudes (±S.E.M.) of the population spike are given 1 h and 48 h after the tetanus (* P<0.05, ** P<0.01 compared to pre-tetanus amplitudes). (b) Time course of LTP in the awake animal. Baseline responses were recorded for 2 days (20 min/day). A tetanus was delivered (arrow) on the second day and responses were monitored for 1 h, and again for 20 min per day for-the next 2 days. +/+ (open circles, n=7), +/− (grey circles, n=11) and −/− (black circles, n=6) mice all showed significant potentiation of the population spike potentiation 1 h after tetanic stimulation, but only +/+ mice maintained this potentiation over the following 48 h.

FIG. 4. In situ hybridisation shows that mRNAs for zif268(in +/+ mice) and lacZ (in −/− mice) are present in the dentate gyrus 1 h following LTP-inducing stimulation of the perforant path (eft and centre panels). [In −/− animals, a portion of the zif268 coding region is replaced by an in-frame lacZ coding sequence.] A hybridisation probe for a region of the zif268 gene transcribed in both −/− and +/+ mice (‘commnon’) revealed similar expression in mice of both genotypes (right panel).

FIG. 5. Spatial navigation in the watermaze. (a) During massed training, all mice took the same amount of time to escape the water on the first trial (Tr1). During acquisition, +/+ mice (open circles, n=9) learned to locate the hidden platform. Although +/− (grey circles, n=9) and −/− mice (black circles, n=9) learned the task, they were slower than +/+ mice. (b) In a probe trial given 48 h later +/+ mice showed a spatial bias for the training quadrant (** P<0.01), but this was not the case with +/− or −/− mice (F<1 for both genotypes) which distributed their time equally between the 4 quadrants. (c) If extended and distributed training was given all mice (+/+, n=11; +/−, n=13; −/−, n 10) learned the task at similar rates and (d) showed retention of the learning in a probe trial given 8 days later (** P<0.01).

FIG. 6. zif268 mutant mice are impaired in conditioned taste aversion, a non-hippocampal associative learning task. The histograms indicate aversion indices (volume sucrose consumed/total volume consumed; mean±S.E.M) for +/+, (n=8), +/−(n=10) and −/− (n=10) mice, following exposure to LiCl or to NaCl. When sucrose was followed by an intraperitoneal injection of lithium chloride, +/+ mice avoided sucrose and preferentially drank water 24 h after conditioning (* P<0.05). In contrast, neither −/− nor +/− mice show a significant aversion to sucrose at 24 h. Control mice in which drinking the novel sucrose solution was followed by a sodium chloride injection (n=4 for each genotype) drank similar volumes of sucrose and water.

FIG. 7. Olfactory discrimination in social transmission of food preference (a) and object recognition (b) is normal at short time intervals but impaired at long intervals in zif268 mutant mice. (a) Histograms representing the preference index exhibited by the observer mice for the scented food eaten by the demonstrator mouse (demonstrated food consumed/total food consumed) at 30 s delay (left) and 24 h delay (right) after interaction between demonstrator and observer mice. All mice (n=10 for each genotype) learned the discrimination when there was minimal delay (* P<0.05), showing a significant preference above chance level for the food eaten by the demonstrator mice. When a 24 h delay was imposed, only +/+ mice (n=10; * P<0.05) retained the memory for the odour. Neither +/− (n=7) nor −/− (n=9) mice showed a significant preference for the flavour they had been exposed to during the interactive period. (b) The histograms show the percent time spent exploring the novel object (time spent exploring the novel object/total time*100) at a 10 minute delay and a 24 h delay in the object recognition task. All mice (n=8 for each genotype) spent significantly more time exploring the novel object at the 10 min delay (* P<0.05). After a 24 delay, +/+ and +/− mice spent more time exploring the novel object (* P<0.05), while −/− mice no longer showed a preference for the novel object.

FIG. 8 shows the densitometric quantification of zif268 mRNA upregulation in an in situ hybridisation experiment similar to that of FIG. 4, but with LTP induced by exposure of the hippocampal slices to chemical induction medium rather than by electrical stimulation.

FIG. 9 shows a Northern blot analysis of total mRNA extracted from cultured cells 30 minutes after chemical induction of LTP, probed for zif268 RNA.

FIG. 10 shows a similar experiment to that shown in FIG. 9, but with varying intervals between induction medium exposure and subsequent cell lysis and mRNA extraction.

FIG. 11 shows a similar experiment to that shown in FIG. 10O, except the Northern blots have been probed for arc RNA.

FIG. 12 shows a preferred embodiment of the assay, in which chemically-induced expression of zif268 is monitored by immunofluorescence, as the proportion of neurons that are zif268-immunoreactive (i.e., cells double-labelled with antibodies to zif268 protein and to the neuron-specific marker protein, NeuN).

FIG. 13 shows an example of an alternative embodiment of the assay, in which chemically-induced expression of zif268 is monitored via the colorimetric detection of β-galactosidase, the gene for which is inserted within the zif268 coding sequence in a “knock-in” transgenic mouse.

FIG. 14 shows that the assay can detect a combination: of drugs (carbachol+isoproterenol, 0.2 μM each) known to enhance LTP: carbachol+isoproterenol, but not carbachol alone, enhances the chemically induced increase in neuronal zif268 immunoreactivity.

FIG. 15 shows that the assay can also detect a drug (PD989590, a MAPK kinase inhibitor) known to inhibit LTP: PD98959 inhibits the chemically-induced expression of zif68mRNA.

FIG. 16 shows that the chemically-induced expression of zif268 mRNA expression is blocked by a combination of drugs that block excitatory glutamatergic synaptic activation.

### DETAILED DESCRIPTION OF THE INVENTION

Detection of Native Gene Expression

The expression of native immediate-early genes, such as zif268 and arc, may be detected directly using any suitable techniques. Preferred are immunofluorescent techniques in which a fluorecently-labelled antibody is used to quantify native gene expression in a cell or cell extract. However, alternative labelling and detection approaches may also be used.

Antibodies against proteins such as zif268 are available in the art; however, the generation of such antibodies and labelling thereof is within the abilities of a person skilled in the art.

Antibodies, as used herein, refers to complete antibodies or antibody fragments capable of binding to a selected target, and including Fv, ScFv, Fab′ and F(ab′)₂, monoclonal and polyclonal antibodies, engineered antibodies including chimeric, CDR-grafted and humanised antibodies, and-artificially selected antibodies produced using phage display or alternative techniques. Small fragments, such as Fv and ScFv, possess advantageous properties for diagnostic and therapeutic applications on account of their small size and consequent superior tissue distribution. Preferably, the antibody is a single chain antibody or scFv.

The antibodies are advantageously labelled antibodies. Such labels may be radioactive labels or radiooopaque labels, such as metal particles, which are readily visualisable in a cell. Preferably, however, they are fluorescent labels or other labels which are visualisable by optical means.

Antibodies may be obtained from animal serum, or, in the case of monoclonal antibodies or fragments thereof, produced in cell culture. Recombinant DNA technology may be used to produce the antibodies according to established procedure, in bacterial or preferably mammalian cell culture. The selected cell culture system preferably secretes the antibody product.

Multiplication of hybridoma cells or mammalian host cells in vitro is carried out in suitable culture media, which are the customary standard culture media, for example Dulbecco's Modified Eagle Medium (DMEM) or RPMI 1640 medium, optionally replenished by a mammalian serum, e.g. foetal calf serum, or trace elements and growth sustaining supplements, e.g. feeder cells such as normal mouse peritoneal exudate cells, spleen cells, bone marrow macrophages, 2-aminoethanol, insulin, transferrin, low density lipoprotein, oleic acid, or the like. Multiplication of host cells which are bacterial cells or yeast cells is likewise carried out in suitable culture media known in the art, for example for bacteria in medium LB, NZCYM, NZYM, NZM, Terrific Broth, SOB, SOC, 2×YT, or M9 Minimal Medium, and for yeast in medium YPD, YEPD, Minimal Medium, or Complete Minimal Dropout Medium.

In vitro production provides relatively pure antibody preparations and allows scale-up to give large amounts of the desired antibodies. Techniques for bacterial cell, yeast or mammalian cell cultivation are known in the art and include homogeneous suspension culture, e.g. in an airlift reactor or in a continuous stirrer reactor, or immobilised or entrapped cell culture, e.g. in hollow fibres, microcapsules, on agarose microbeads or ceramic cartridges.

Large quantities of the desired antibodies can also be obtained by multiplying mammalian cells in vivo. For this purpose, hybridoma cells producing the desired antibodies are injected into histocompatible mammals to cause growth of antibody-producing tumours. Optionally, the animals are primed with a hydrocarbon, especially mineral oils such as pristane (tetramethyl-pentadecane), prior to the injection. After one to three weeks, the antibodies are isolated from the body fluids of those mammals. For example, hybridoma cells obtained by fusion of suitable myeloma cells with antibody-producing spleen cells from Balb/c mice, or transfected cells derived from hybridoma cell line Sp2/0 that produce the desired antibodies are injected intraperitoneally into Balb/c mice optionally pre-treated with pristane, and, after one to two weeks, ascitic fluid is taken from the animals.

The foregoing, and other, techniques are discussed in, for example, Kohler and Milstein, (1975) Nature 256:495-497; U.S. Pat. No. 4,376,110; Harlow and Lane, Antibodies: a Laboratory Manual, (1988) Cold Spring Harbor, incorporated herein by reference. Techniques for the preparation of recombinant antibody molecules are described in the above references and also in, for example, EP 0623679; EP 0368684 and EP 0436597, which are incorporated herein by reference.

The cell culture supernatants are screened for the desired antibodies, preferentially by immunofluorescent staining of cells expressing the desired target by immunoblotting, by an enzyme immunoassay, e.g. a sandwich assay or a dot-assay, or a radioimmunoassay.

For isolation of the antibodies, the immunoglobulins in the culture supernatants or in the ascitic fluid may be concentrated, e.g. by precipitation with ammonium sulphate, dialysis against hygroscopic material such as polyethylene glycol, filtration through selective membranes, or the like. If necessary and/or desired, the antibodies are purified by the customary chromatography methods for example gel filtration, ion-exchange chromatography, chromatography over DEAE-cellulose and/or (immuno-) affinity chromatography, e.g. affinity chromatography with the target molecule or with Protein-A.

Antibodies generated according to the foregoing procedures may be cloned by isolation of nucleic acid from cells, according to standard procedures. Usefully, nucleic acids variable domains of the antibodies may be isolated and used to construct antibody fragments, such as scFv.

Recombinant DNA technology may be, used to improve the antibodies useful in the invention.

Antibodies may moreover be generated by mutagenesis of antibody genes to produce artificial repertoires of antibodies. This technique allows the preparation of antibody libraries, as discussed further below; antibody libraries are also available commercially. Hence, the present invention advantageously employs artificial repertoires of immunoglobulins, preferably artificial ScFv repertoires, as an immunoglobulin source.

Suitable fluorophores are known in the art, and include chemical fluorophores and fluorescent polypeptides, such as GFP and mutants thereof (see WO 97/28261). Chemical fluorophores may be attached to immunoglobulin molecules by incorporating binding sites therefor into the immunoglobulin molecule-during the synthesis thereof.

Preferably, the fluorophore is a fluorescent protein, which is advantageously GFP or a mutant thereof. GFP and its mutants may be synthesised together with the immunoglobulin or target molecule by expression therewith as a fusion polypeptide, according to methods well known in the art. For example, a transcription unit may be constructed as an in-frame fusion of the desired CYFP and the immunoglobulin or target, and inserted into a vector as described above, using conventional PCR cloning and ligation techniques.

Antibodies may be labelled with any label capable of generating a signal. The signal may be any detectable signal, such as the induction of the expression of a detectable gene product. Examples of detectable gene products include bioluminescent polypeptides, such as luciferase and GFP, polypeptides detectable by specific assays, such as p-galactosidase and CAT, and polypeptides which modulate the growth characteristics of the host cell, such as enzymes required for metabolism such as HIS3, or antibiotic resistance genes such as G418. In a preferred aspect of the invention, the signal is detectable at the cell surface. For example, the signal may be a luminescent or fluorescent signal, which is detectable from outside the cell and allows cell sorting by FACS or other optical sorting techniques.

Preferred is the use of optical immunosensor technology, based on optical detection of fluorescently-labelled antibodies. Immunosensors are biochemical detectors comprising an antigen or antibody species coupled to a signal transducer which detects the binding of the complementary species (Rabbany et al., 1994 Crit Rev Bidmed Eng 22:307-346; Morgan et al., 1996 Clin Chem 42:193-209). Examples of such complementary species include the antigen zif268 and the anti-zif268 antibody. Immunosensors produce a quantitative measure of the amount of antibody, antigen or hapten present in a complex sample such as serum or whole blood (Robinson 1991 Biosens Bioelectron 6:183-191).

The sensitivity of immunosensors makes them ideal for situations requiring speed and accuracy (Rabbany et al., 1994 Crit Rev Biomed Eng 22:307-346).

Detection techniques employed by immunosensors include electrochemical, piezoelectric or optical detection of the immunointeraction (Ghindilis et al., 1998 Biosens Bioelectron 1:113-131). An indirect immunosensor uses a separate labelled species that is detected after binding by, for example, fluorescence or luminescence (Morgan et al., 1996 Clin Chem 42:193-209). Direct immunosensors detect the binding by a change in potential difference, current, resistance, mass, heat or optical properties (Morgan et al., 1996 Clin Chem 42:193-209). Indirect immunosensors may encounter fewer problems due to non-specific binding (Attridge et al., 1991 Biosens Bioelecton 6:201-214; Morgan et al., 1996 Clin Chem 42:193-209).

Reporter Gene Construction

Reporter genes may be constructed according to standard techniques known in the art. In general, the reporter may be either a coding sequence which is heterologous to, and driven by, the selected regulatory sequence (see below); or a modification of the coding sequence normally associated with the selected regulatory sequence, such that expression thereof is detectable; or, in some cases, the natural coding sequence normally associated with the selected regulatory sequence may be usable as a reporter gene, if expression of that sequence is or gives rise to a detectable event.

The reporter gene may be directly or indirectly induced by events related to LTP induction. That is, the expression of an immediate early gene which is associated with LTP may be used as a second signal to induce expression of a reporter gene. This is facilitated by the fact that several immediate early gene products are transcription factors, such as zif268, or are otherwise involved in the regulation of gene transcription. In this instance, the reporter gene is operatively linked to sequences which are responsive to the expression of immediate early gene products. For example, the reporter gene may be under the control of a zif268-responsive enhancer, e.g. GFP or luciferase under control of one or more egr response element (ERE) such as those in the promoters of the TGFβ1 gene (Levkovitz, Y., et al., (2001), J. Neurosci. 21:45-52) or the PTEN tumour suppressor gene (Virolle, T., et al., (2001), Nature Cell Biol. 3:11241128).

In a preferred aspect of the invention, the reporter gene may be incorporated into a vector designed for replication of DNA, and/or transient or permanent transformation of cells allowing expression of the reporter gene.

As used herein, vector (or plasmid) refers to discrete elements that are used to introduce heterologous DNA into cells for either expression or replication thereof. Selection and use of such vehicles are well within the skill of the artisan. Many vectors are available, and selection of appropriate vector will depend on the intended use of the vector, i.e. whether it is to be used for DNA amplification or for DNA expression, the size of the DNA to be inserted into the vector, and the host cell to be transformed with the vector. Each vector contains various components depending on its function (amplification of DNA or expression of DNA) and the host cell for which it is compatible. The vector components generally include, but are not limited to, one or more of the following: an origin of replication, one or more marker genes, an enhancer element, a promoter, a transcription termination sequence and a signal sequence.

Both expression and cloning vectors generally contain nucleic acid sequences that enable the vector to replicate in one or more selected host cells. Typically in cloning vectors, this sequence is one that enables the vector to replicate independently of the host chromosomal DNA, and includes origins of replication or autonomously replicating sequences. Such sequences are well known for a variety of bacteria, yeast and viruses The origin of replication from the plasmid pBR322 is suitable for most Gram-negative bacteria, the 2μ plasmid origin is suitable for yeast, and various viral origins (e.g. SV 40, polyoma, adenovirus) are useful for cloning vectors in mammalian cells. Generally, the origin of replication component is not needed for mammalian expression vectors unless, these are used in mammalian cells competent for high level DNA replication, such as COS cells.

Most expression vectors are shuttle vectors, i.e. they are capable of replication in at least one class of organisms but can be transfected into another class of organisms for expression. For example, a vector is cloned in E. coli and then the same vector is transfected into yeast or mammalian cells even though it is not capable of replicating independently of the host cell chromosome. DNA can alternatively be amplified by PCR and be directly transfected into the host cells without any replication component.

Advantageously, an expression and cloning vector may contain a selection gene also referred to as a selectable marker. This gene encodes a protein necessary for the survival or growth of transformed host cells grown in a selective culture medium. Host cells not transformed with the vector containing the selection gene will not survive in the culture medium. Typical selection genes, encode proteins that confer resistance to antibiotics and other toxins, e.g. ampicillin, neomycin, methotrexate or tetracycline, complement auxotrophic deficiencies, or supply critical nutrients not available from complex media.

As to a selective gene marker appropriate for yeast, any marker gene can be used which facilitates the selection for transformants due to the phenotypic expression of the marker gene. Suitable markers for yeast are, for example, those conferring resistance to antibiotics G418, hygromycin or bleomycin, or provide for prototrophy in an auxotrophic yeast mutant, for example the URA3, LEU2, LYS2, TRP1, or mHS3 gene.

Since the replication of vectors is conveniently done in E. coli, an E. Coli genetic marker and an E. coli origin of replication are advantageously included. These can be obtained from E. coli plasmids, such as pBR322, Bluescript© vector or a pUC plasmid, e g., pUC18 or pUC19, which contain both E. coli replication origin and E. coli genetic marker conferring resistance to antibiotics, such as ampicillin.

Suitable selectable markers for mammalian cells are those that enable the identification of cells which have taken up the vector, such as dihydrofolate reductase (DHFR, methotrexate resistance), thymidine kinase, or genes conferring resistance to G418 or hygromycin. The mammalian cell transformants are placed under selection pressure which only those transformants which have taken up and are expressing the marker are uniquely adapted to survive. In the case of a DHFR or glutamine synthase (GS) marker, selection pressure can be imposed by culturing the transformants under conditions in which the pressure is progressively increased, thereby leading to amplification (at its chromosomal integration site) of both the selection gene and the lied DNA that encodes the reporter gene. Amplification is the process by which genes in greater demand for the production of a protein critical for growth, together with closely associated genes which may encode a desired protein, are reiterated in tandem within the chromosomes of recombinant cells. Increased quantities of desired protein are usually synthesised from thus amplified DNA.

Expression and cloning vectors usually contain a promoter that is recognised by the host organism and is operably linked to the reporter construct. Such a promoter may be inducible or constitutive, but will in any case be subject to regulation by a regulatory sequence as defined herein. The promoters may be operably linked to DNA encoding the reporter gene by removing the promoter from the source DNA and inserting the isolated promoter sequence into the vector. Both the native promoter sequence normally associated with the reporter and many heterologous promoters may be used to direct expression of the reporter gene. The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences.

Preferably, the promoter is itself responsive to modulation by events associated with LTP. Thus, it may be a promoter derived from a gene whose expression is modulated in association with LTP, or it may be a promoter which is modulated by the gene product of a gene which is whose expression is modulated in association with LTP.

Transcription of a reporter gene by-higher eukaryotes may be modulated by inserting an enhancer sequence into the vector. This permits a promoter which is not normally responsive to events associated with LTP induction to be rendered so responsive. Enhancers are relatively orientation and position independent. Many enhancer sequences are known from immediate early genes which are subject to LTP-associated modulation and can be selected by a person skilled in the art according to need.

Eukaryotic expression vectors may also contain sequences necessary for the termination of transcription and for stabilising the mRNA. Such sequences are commonly available from the 5′ and 3′ untranslated regions of eukaryotic or viral DNAs or cDNAs. These regions contain nucleotide segments transcribed as polyadenylated fragments in the untranslated portion of the mRNA encoding the reporter gene.

An expression vector includes any vector capable of expressing a reporter gene as described herein. Thus, an expression vector refers to a recombinant DNA or RNA construct, such as a plasmid, a phage, recombinant virus or other vector, that upon introduction into an appropriate host cell, results in expression of the cloned DNA. Appropriate expression-vectors are well known to those with ordinary skill in the art and include those that are replicable in eukaryotic and/or prokaryotic cells and those that remain episomal or those which integrate into the host cell genome. For example, DNAs encoding a reporter gene may be inserted into a vector suitable for expression of cDNAs in mammalian cells, e.g. a CMV enhancer-based vector such as pEVRF (Matthias, et al., (1989) NAR 17, 6418).

Useful for practising the present invention are expression vectors that provide for the transient or stable expression of DNA encoding reporter genes in mammalian cells. Transient expression usually involves the use of an expression vector that is able to replicate efficiently in a host cell, such that the host cell accumulates many copies of the expression vector, and, in turn, synthesises high levels of the reporter gene when stimulated to do so.

Construction of vectors according to the invention employs conventional ligation techniques. Isolated plasmids or DNA fragments are cleaved, tailored, and religated in the form desired to generate the plasmids required. If desired, analysis to confirm correct sequences in the constructed plasmids is performed in a known fashion. Suitable methods for constructing expression vectors, preparing in vitro transcripts, introducing DNA into host cells, and performing analyses for assessing reporter gene expression and function are known to those skilled in-the art. Gene presence, amplification and/or expression may be measured in a sample directly, for example, by conventional Southern blotting, Northern blotting to quantitate the transcription of mRNA, dot blotting (DNA or RNA analysis), or in situ hybridisation, using an appropriately labelled probe which may be based on a sequence provided herein. Those skilled in the art will readily envisage how these methods may be modified, if desired.

Downstream Optical Reporters

Induction of LTP may also be monitored optically via other phenonema associated wt physiological expression of the potentiation. Thus, for example, optical markers of synaptic vesicle cycling in transmitter release, such as fluorescent lipophillic dyes FM1-43 or FM4-64 (Cochilla. A. J., et al., (1999). Annu. Rev. Neurosci. 22: 0.1-10) or fluorescent labelled antibodies directed against luminal epitopes of synaptic vesicle proteins (Malgaroli, A., et al., (1995) Science 268 1624-1628), can be used to detect increased rates of spontaneous transmitter release after LTP induction (e.g., Magaroli, A., et al., op. cit.).

Transformation of Cells

Cells suitable for performing an assay according to the invention are preferably higher eukaryote cells derived from a multicellular organism, and advantageously are mammalian cells. The preferred cell types are neural cells, which may be primary cultures of cells of a neural lineage, or immortalised cell lines of a neural lineage.

Cells may be transformed by any suitable technique available in the art. A number of techniques, such as calcium phosphate precipitation and electroporation are described in Sambrook et al., (1989) Molecular Biology; A Laboratory Manual, Cold Spring Harbor, which is incorporated herein by reference. The preferred number of cells is from about 1 to about 5×10⁵ cells, or about 2×10² to about 5×10⁴ cells. In these methods the predetermined amount or concentration of the molecule to be tested is typically based upon the volume of the sample, or be from about 1.0 μM to about 20 μM, or from about 10 nM to about 500 μM.

The invention also encompasses the performance of the assay in transgenic animals, preparable for example by pronuclear microinjection or by the preparation of ES cell chimeras, according to established techniques.

Exposure of Cells to Test Compounds

Typically the contacting is effected from about 1 minute to about 24 hours, preferably from about 2 minutes to about 1 hour. Also the contacting is typically effected with more than one predetermined amount of the molecule to be tested. The molecule to be tested in these methods can be a purified molecule, a mixture of molecules or a homogenous sample.

Cells or tissues to be assayed are preferably incubated with the test compound or mixture in an isotonic salt solution, such as Hank's balanced salt solution (HBSS), or artificial cerebrospinal fluid (ACSF) for the period of the exposure. The test compound may be added to the cells by simple addition, in or, preferably, by inclusion with an LTP-inducing stimulus such as a “potentiation medium” (also referred to as “induction medium”) which includes the compound(s) to be tested. Such potentiation medium may, for example, constitute an isotonic salt solution to which glutamate receptor agonists are added, or in which potassium concentration is elevated, with or without addition of tetraethylammonium, elevated calcium, and/or reduced magnesium. The medium is then advantageously changed, to a medium which supports growth of the cell type in question. In the case of neural cells, a neural growth medium such as NeuroBasal (Gibco BRL) may be used. The cells are then assayed for reporter gene expression as described below.

Compounds or mixtures of compounds are advantageously brought into contact with cells in the context of a high-throughput screening assay. Thus, a number of test systems may be contacted with different compounds or mixtures of compounds, or different amounts of compounds or mixtures, at the same time. The effects of the addition of the compounds are measured by following the detectable signal selected for the test system in use, according to the present invention.

LTP may be induced by chemical or other means, and the activity of the test compound(s) in modulating LTP monitored by means of the assay of the invention. For example, LTP may be induced chemically, as described above and in more detail below, or by field electrical stimulation. Thus, for example, the cells may be arranged in a multiwell plate and stimulated using a grid to supply a suitable electrical field, with LTP induction measured by optical screening as described.

Generation of a Detectable Signal

A detectable signal may be generated in any one of a number of ways, depending on the nature of the reporter gene employed in the method of the invention. For example, the detectable signal may be a luminescent or fluorescent signal, or may be a signal generated as a result of enzymatic activity.

A preferred fluorescent signal is provided by GFP expression. GFP is a fluorescent polypeptide which produces a fluorescent signal without the need for a substrate or cofactors. GFP expression and detection techniques are well known in the art, and kits are available commercially, for example from Clontech. GFP expression may be assayed in intact cells without the need to lyse them or to add further reagents.

Luciferase may also be used as a basis for an assay. Luciferase expression is known in the art, and luciferase expression and detection kits are available, commercially from Clontech (Palo Alto, Calif.). The presence of luciferase is advantageously assessed by cell lysis and addition of luciferin substrate to the cells, before monitoring, for a luminescent signal by scintillation counting.

Enzyme-based assays are conducted in a manner similar to a luciferase-based assay, except that the detection is not necessarily via luminescence. The detection technique will depend on the enzyme, and may therefore be optical (such as in the case of β-galactosidase).

The invention is Her described below, for the purposes of illustration only, in the following examples.

### Examples

### Example 1

Zif268 Expression is Required for LTP

In order to illustrate-the relationship between LTP and zif268 expression, zif268−/− knockout mice were analysed for LTP under a variety of physiological conditions, as assessed by standard physiological tests for LTP.

Animals

Mice carrying a targeted inactivation of the Zif268 gene were generated by Topilko et al., Mol. Endocrin. 0.12, 107-122 (1998), using 129/SV ES cells injected into C57BL/6J. blastocysts. Mice were backcrossed onto a C5713L/6J background. The mutation involved the; insertion of a lacZ-neo cassette containing a polyadenylation site between the promoter and its coding sequence, which prevented transcription of the gene. In addition a frameshift mutation was introduced into the coding sequence at the level of an NdeI restrictions sited corresponding to the beginning of the DNA-binding domain. This mutation differed from that previously described by Lee et al., J. Biol. Chem., 270, 9971-20-9977 (1995), who inserted a neo cassette at the same NdeI site. Age-matched (2-8 month old) +/+, +/− and −/− littermates were used throughout, with experimenters blind to genotype. Most mice were tested in more that one behavioural task, with the sequence of tasks randomised to eliminate interference. A: subset of the same mice was subsequently used for electrophysiological studies. All were performed in strict accordance with recommendations of the EU (86/609/EEC), the French National Committee (87/848) and the U.K. Home Office Animals (Scientific Procedures) Act, 1986.

Spontaneous Alternation

Mice explored the T-maze freely (with all arms open) for 10-20 min over two days during the habituation period. The maze floor was covered with sawdust that was redistributed after each run to reduce the possibility of using olfactory cues. Mice were placed in the start box for 30 s and then given a forced choice run (randomly assigned) by blocking access to the other arm. On entering the arm, they were held there for 30s. Following the forced choice run, mice were immediately put back in the start box and held there for 30 s before being released into the maze, where they had access to both arms. Each mouse was given 4-8 trials per day for 3 days. Alternation was expressed as the percent number of trials on which mice chose the opposite arm to the one which they entered during the initial forced choice. As the alternation score could only fall between 0-100%, data were subjected to, angular transformation before being analysed with Student's t-test.

Spatial Navigation

The water maze was 1.5 m diameter, containing a 10 cm diameter escape platform. The platform position for each mouse was fixed in the centre of one of the training quadrants. For massed training, mice were given 2 blocks of 4 trials with the hidden platform located in the centre of the maze during a 1 day habituation period. Trials were given on the following day, with 5 blocks of 5 trials. The maximum swim time for each trial was 60 s, with an inter-trial interval of 120 s, during which the mouse remained on the platform. The 5 trials were run consecutively and the interval between blocks was 15 min. Swim time and inter-trial interval were chosen to avoid mice becoming fatigued. A 60 s probe trial with the platform removed was run 48 h post-training.

The distributed training protocol was similar to that for massed training with the exception that the mice were given 1 block of 4 pre-training trials. During the acquisition phase, mice were tested on 2blocks of 4 trials a day for 10 days. The interval between trials was 60 s on the platform and 5 h between blocks. The maximum swim time was 90 s. A 90 s probe trial was given 8 days following the end of acquisition. Analysis of variance was conducted on the latency to escape the water during the acquisition phase, and the time spent in each quadrant during the probe trial.

Conditioned Taste Aversion

Water-deprived mice were trained for 3 days to drink ad libitum from 2 identical water bottles presented in the home cage for 30 min per day. Water bottles were weighed to establish the volume consumed over the 30 min period. On the conditioning day, mice received 15% sucrose in 2 identical bottles for 30 min. One hour following removal of sucrose, mice were either injected with 0.9% saline, or 0.3M LiCl (10% body weight i.p.). Twenty four and 48 h after conditioning, mice were offered the choice between water and sucrose for 30 min. The aversion index was calculated as the volume of sucrose consumed divided by the total volume consumed, expressed as a percentage; a lower index means a stronger aversion. Data were subject to angular transformation before statistical analyses: Student's t-tests were used to analyse differences from chance (an aversion index of 0.5) and the three test groups were compared using one-way Analysis of variance.

Social Transfer of Food Preference

Demonstrator mice were habituated to test cages for several days. They were deprived of food the night before being presented with a novel taste. On the test day, demonstrators were given crushed standard laboratory pellets scented with coriander (0.3%) or bitter cocoa (2.0%) for 2 h; half the mice were given coriander and half cocoa. Observer mice, which had also been food deprived for 0.22 h per. day for 2 days and habituated to the test cages, were then given a 20 min period to interact with the demonstrators. Test mice were either placed in the test cages immediately after the interaction (30 s time point) or 24 h later, when they were allowed free access to both scented foods for 20 min. Both male and female mice were used in this task, and as there was no obvious difference in, performance between the sexes, all animals were grouped for final analysis. The amount of each type of food consumed was calculated by weighing before and after the test. A preference ratio, expressed as a percentage, was calculated by dividing the amount of the demonstrated food eaten by the total amount of food eaten. A Wilcoxon Ranked Scores test was carried out, comparing the preference ratio with 50% (no preference) for each genotype at each time point.

Object Recognition

Mice were habituated to the testing box (30 cm×20 cm×10 cm) for 20 min per day for 3-4 days before testing commenced. A range of objects of similar-size but varied shape and colour were used. Two objects were placed in the box in fixed positions and mice were allowed to explore freely for two 10 min sessions, separated by a 10 min break in the home cage. Perimeters were drawn around the two objects and the time spent with both forelimbs within a perimeter, orientated towards an object was quantified. Active exploration of the objects tended to involve sniffing and touching. During the test phase (either 10 min or 24 h later) one of the objects was replaced by a novel object. Mice were returned to the box and the time spent exploring the two objects was again recorded. Data are presented as the proportion of time spent exploring each object as a percentage of the total exploration time. A relatively greater amount of time spent exploring the novel object was taken to indicate that mice recognised the familiar object from 10 min or 24 h earlier. Students paired t-test comparing per-cent exploration of novel and familiar objects were used to assess recognition learning.

Surgery and Electrophysiology

Acute, non-recovery experiments were performed in-wildtype and homozygous mice under urethane anaesthesia (1.8 g/kg i.p.). Mice were placed in a stereotaxic frame to, allow placement of a concentric bipolar stimulating electrode (p.d. 100 μm, Rhodes Electromedical) positioned in the perforant path (2.7-3.1 mm lateral of lambda), and a glass micropipette recording electrode in the hilus of the ipsilateral dentate gyrus (2.0 mm posterior and 1.5-1.6 mm lateral of bregma). Pairs of pulses with inter-pulse intervals in the range of 10-100 ms were used to study paired-pulse facilitation at two stimulus intensities: a low-intensity stimulus, evoking an EPSP with 10% maximum amplitude and a high-intensity stimulus evoking a population spike of approximately 1 mV. Inter-pulse intervals-were tested in triplicate and averaged for analysis. Pre-tetanus test responses were evoked by 50 or 60 Us monophasic pulses (100-300 μA) at 1 per 30s, until a stable baseline was achieved. The pulse width was doubled during tetanic stimulation, which consisted of 6 series of 6 trains of 6 stimuli: at 400 Hz, 100 ms between trains, 20 s between series.

Long-term plasticity was studied in the freely-moving mouse. Detailed methods for recording from awake mice are described by Davis et al., Neurosci. Methods 75, 75-80 (1997). Briefly, mice were anaesthetised with sodium pentobarbital (60 mg/kg i.p.) and placed in a stereotaxic frame. A concentric bipolar stimulating electrode was positioned in the perforant path and a 65 ∥m nichrome recording wire positioned in the hilus of the ipsilateral dentate gyrus. A silver reference electrode was positioned over the surface of the contralateral cortex. Electrode positions were adjusted to maximise evoked field potentials, and electrodes were fixed with dental cement. Animals were housed individually and allowed to recover for at least 7 days before being connected for recording. Mice were habituated to the recording chamber for at least 3 days before receiving tetanic stimulation, each animal being tested in the same chamber throughout.

Pre-tetanus test responses were evoked at a frequency of 1 per 30 s for 20 ml periods on consecutive days. Monophasic pulses (80 μs) of 26-250 μA were used for test stimulation, the intensity for each mouse being set to evoke a 1-3 mV population spike. If responses were stable during two successive 20 min test periods in 24 h, tetanic stimulation was delivered to the perforant path. Each tetanus consisted of 6 series of 6 trains of 6 stimuli at 400 Hz, 100 ms between trains, 20 s between series. The pulse width was increased from 80 μs to 100 μs during tetani. Responses were followed for a subsequent 60 min on the day of the tetanus, and then for 20 min periods 24 and 48 h post-tetanus. Data are expressed as mV change in amplitude (mean±S.E.M) relative to the mean of the test responses measured over the 10 min prior to tetanic stimulation. Student's t-test was used to compare mean levels of potentiation over the periods indicated.

In Situ Hybridisation

One hour after tetanic stimulation, brains were removed, frozen on dry-ice and stored at −70° C. Fourteen μm thick sections were cut on a cryostat, mounted poly-L-lysine coated glass slides and stored at −70° C. In situ hybridisation was performed essentially as described by Wisden et al., Neuron 4, 603-604 (1990). Briefly, sections were thawed at room temperature, fixed in 4% paraformaldehyde, acetylated in 1.4% triethanolamine and 0.25% acetic anhydride, dehydrated through graded ethanol solutions and delipidated in chloroform. Sections were hybridised overnight at 42° C. in 100 ml buffer containing 50% formamide, 4×SSC (150 mM sodium chloride/50 mM sodium citrate), 10% dextran sulphate, 5× Denhardt's, 200 mg/ml acid alkali cleaved salmon testis DNA, 100 mg/ml long chain polyadenylic acid, 25 AM sodium phosphate (pH 7.0), 1 mM sodium pyrophosphate and 100 000 cpm radiolabelled probe (˜1 ng/ml) under parafilm coverslips. Sections were washed in 1×SSC at 55° C. (30 min), 0.1×SSC at room temperature (5 min) and dehydrated in 70% and 95% ethanol. Sections were then exposed to autoradiographic film. ³⁵S-AT? end labelled probes (NEN) were generated using terminal deoxynucleotidyl transferase (Promega) according to manufacturer's instructions. A 50 fold excess of unlabelled oligonucleotide was used as negative control. Oligonucleotides of unique sequence were supplied by Oswel (Southampton, UK). Probe sequences were: zif268, CCGTGGCTCAGCAGCATCATCTCCTCCAGTTTGGGGTAGTTGTCC, complementary to nucleotides spanning amino acid 2-16 (Milbrandt, J., (1987) Science 85, 7857-7861). LacZ, TTGGTGTAGATGGGCGCATCGTAACCGTGCA TCTGCCAGTTTGAG, complementary to nucleotides 261-305. The probe against the 5′ UTR of zif268, common to both wildtype and mutant mice, was GGGTTACATGCGGGGTGCAGGGGCACACTGCGGGGAGT, complementary to nucleotides 90-128 upstream of the AUGstart codon.

Histology

Mice were perfused with 4% paraformaldehyde in PBS and brains post-fixed overnight. Free-floating sections (40 μm) were washed 4 times in PBS (0.1 M) for 10 min each: wash. Cresyl violet was used as a general cell stain. Anti-NeuN (1/6000 dilution, Chemicon) was used to mark healthy and viable cells. Anti-Synaptophysin (1/100, Boehringer Mannheim) was used to mark presynatpic boutons and anti-Parvalbumin (1/1000, Sigma) to label neuronal calcium binding proteins. Non specific epitopes were blocked by incubation in 10% normal goat serum and 0.1% triton X-100,in PBS for 1 h. Sections were incubated with the primary antibodies for 48 h at room temperature and then washed 3 times in PBS. Secondary antibody was applied for 1 h at room temperature, and the sections then washed a fiber 3 times in PBS. Immunostaining was visualised using an ABC elite system (Vector Labs) and a VIP revelation kit (Biosystems).

Hippocampal Anatomy in Zif268 −/− Mice

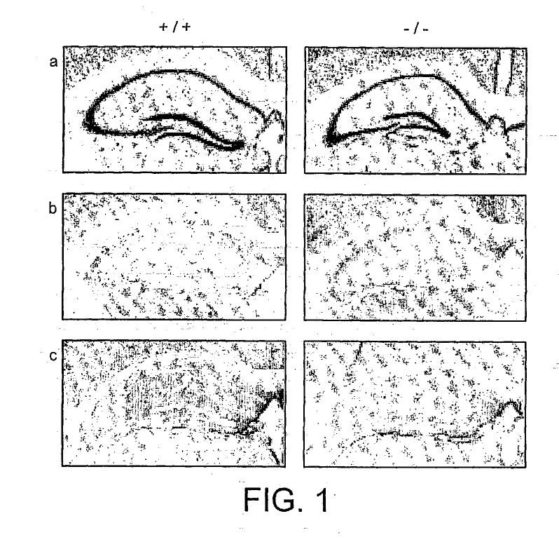

We have used several histochemical and immunohistochemical markers to examine hippocampal anatomy in control and mutant mice. Nissl staining (not shown) and NeuN immunoreactivity (which labels a neuronal-specific DNA-binding protein (Wolf, H. K. et al., (1996) J. Histochem. Cytochem. 44, 1167-1171); FIG. 1a) were indistinguishable in wildtype (+/+) and homozygous mutant (−/−) mice. Parvalbumin labels a calcium binding protein in a subpopulation of GABAergic interneurons in the hippocampus (Kosaka, et al., (1987) Brain Res. 419, 119-130), again showing similar staining in both +/+ and −/− mice (FIG. 1b). Synaptophysin immunoreactivity in synaptic layers also appeared normal in −/− mice (FIG. 1e). Thus the basic hippocampal anatomy was not affected by the zif68 mutation.

Short-Term Plasticity and LTP in Anaesthetised Mice

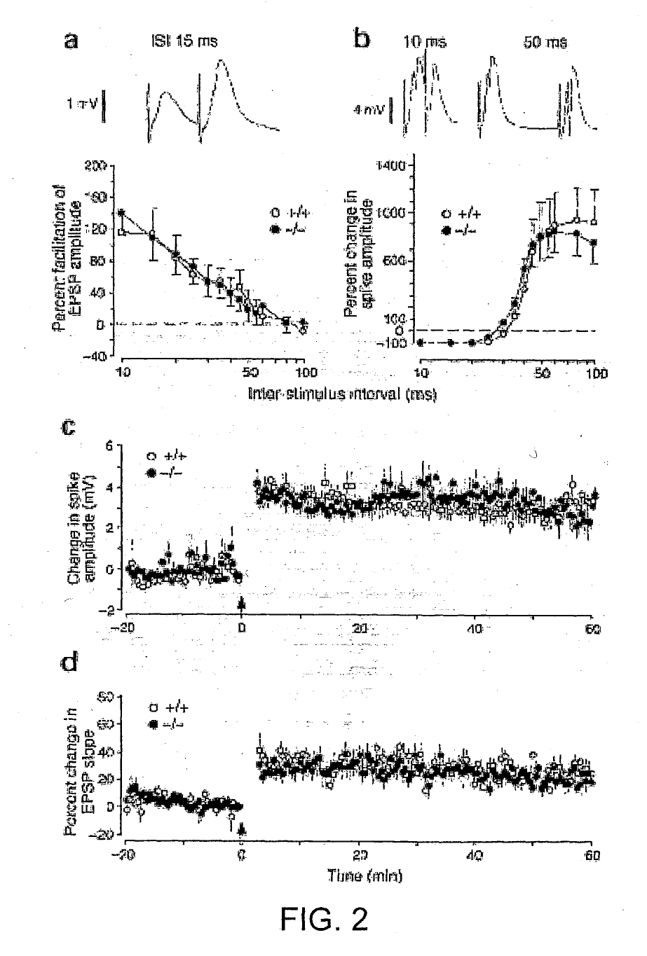

We measured LTP and short-term plasticity of both the field EPSP (FEPSP) and population spike in urethane anaesthetised mice. Paired-pulse stimulation (inter-pulse interval 10-100 ms) at intensities sub-threshold for a population spike resulted in a characteristic facilitation of the FEPSP, maximal at inter-pulse intervals of 10-20 ms, in both +/+ and −/− mice (FIG. 2a). Paired-pulse facilitation of FEPSP at short inter-pulse intervals is attributable to presynaptic mechanisms, involving an accumulation of calcium in presynaptic terminals and increased transmitter release to the second stimulus of a pair (Katz B. & Miledi, R (1968) J. Physiol. (Lond) 195, 481-492). Paired-pulse stimulation at intensities just above threshold for evoking a population spike resulted in complete suppression of the population spike at short inter-stimulus intervals (10-25 ms) and spike facilitation at longer intervals in both +/+ and −/− mice (FIG. 2b). Spike facilitation peaked at inter-pulse intervals of 60-80 ms in all mice tested. This profile of spike depression at short inter-stimulus intervals followed by facilitation at longer intervals most likely reflects recurrent inhibition and disinhibition and is therefore in part a postsynaptic phenomenon. Thus, short-term presynaptic plasticity and network excitability in the dentate gyrus appear to be normal in zif268 mutant mice.

Following tetanic stimulation, both, genotypes showed significant and similar potentiation of the population; spike 50-60 min post-tetanus (2.8±0.5 mV increase in +/+ and 2.8±0.7 mV in −/− mice, P<0.05 vs. test responses; FIG. 2c). LTP of the fBPSP slope was also similar in both genotypes (23±5.3% in +/+ and 21±6.1% in −/− mice, P<0.05 with respect to baseline; FIG. 2d). In summary, we were unable to demonstrate any effect of the zif268 mutation on LTP measured over 1 h in anaesthetised mice.

Long-Lasting Synaptic Plasticity in the Freely Moving Mice

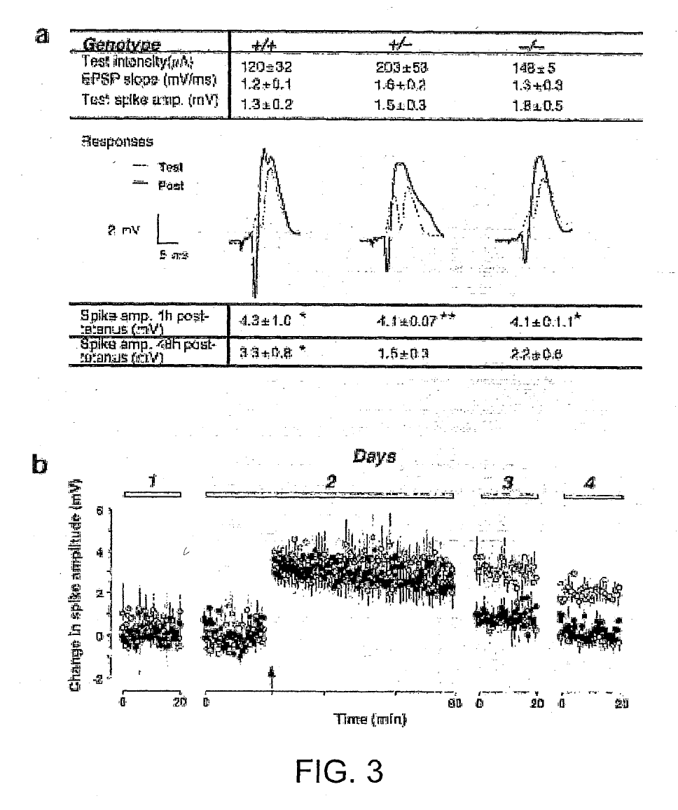

We investigated LTP over several days in freely moving mice. Single stimuli delivered to the perforant path evoked positive-going fEPSPs with negative-going population spikes in the hilus of zif268 +/+, +/− and −/− mice (FIG. 3a). The test stimulus intensities required to evoke responses with comparable FEPSP slopes and population spike amplitudes were not different between genotypes (FIG. 3a). Following two days of baseline recording, tetanic stimulation of the perforant path induced significant potentiation of the population spike in all genotypes (P<0.05 vs. control for spike amplitude 50-60 min post tetanus in +/+ and −/− mice, P<0.01 in +/− mice). The decrease in the latency of the population spike after tetanic stimulation made it impracticable to compare the initial slope of the FEPSP before and after LTP induction (see FIG. 3a). Thus, as in anaesthetised mice, potentiation in mutants was normal in the first hour following induction. Wildtype mice maintained significant spike potentiation 24 h and 48 h following the tetanic stimulation (P<0.05 vs. control). In contras neither +/− nor −/− mice supported significant potentiation at these time points (P>0.06 vs. control values, P<0.05 vs. +/+ mice; see FIG. 3b). Thus the zif268 gene is necessary for the expression of the later phases of hippocampal LTP.

Lack of Expression of Zif268 in the Hippocampus

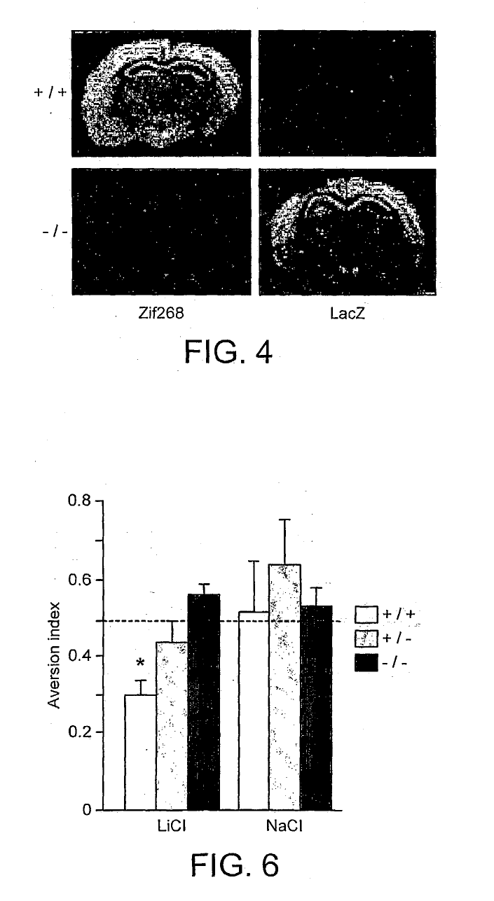

We examined levels of zif268 mRNA in +/+ and −/− mice by in situ hybridisation. The results confirmed a complete absence of zif268 mRNA in the −/− mice, whereas in the +/+ mice there was normal and LTP-inducible expression of zif268 (FIG. 4). The construct used to inactivate zif68 in the mutant mice (Topilko, P. (1998) et al. Mol. Endocrin. 12, 107-122) involved the insertion of a lacZ cassette downstream of the promoter; in situ hybridisation demonstrated normal constitutive and LTP-regulated expression of the lacZ gene in-the −/− mice (FIG. 4), suggesting that the activity of the zif268 promoter was not affected by the mutation. This was confirmed by in situ hybridisation with a common probe derived from the 5′-untranslated region of zif268 and located upstream of the site of insertion of lacZ. The corresponding sequence is expected to be present on both zif268 and lacZ mRNAs. Indeed, the common probe detected equivalent levels of LTP-inducible mRNAs in-both +/+ and −/− mice (FIG. 4).

Learning and Memory Deficits in zif268 Mutant Mice

We next examined whether the deficits in synaptic plasticity in zif268 mutant mice, particularly evident in the late phases of LTP, were paralleled by corresponding deficits in learning and memory. We first evaluated short-term, working memory by testing spontaneous alternation in a T-maze, a spatial behaviour that depends on the integrity of the hippocampus (Gerlai, R (1998) Behav. Brain Res. 95, 91-101). We found that all three genotypes showed normal alternation between the arms visited on the first and second runs of a trial when there was a 1 min delay imposed between runs (+/+: 75±6.8%; +/−: 76:±3.0% and +/−: 69±5.1% alternation, P<0.05 vs. chance in each case). Mice were also tested with a 10 minute delay between runs; again there was no difference in the levels of alternation between genotypes (+/+: 70±4.4%; +/−: 71±7.7% and −/−: 60±6.1%; n=12 per group). All mice made a similar mean number of entries into the arms during the period of habituation to the T-maze, indicating a similar level of exploratory behaviour and locomotion. These data indicate that this type of spatial working memory can occur in the absence of the expression, of zif268. In order to assess the role of zif268 in longer lasting forms of spatial and associative memory we trained mice in four further behavioural tasks.

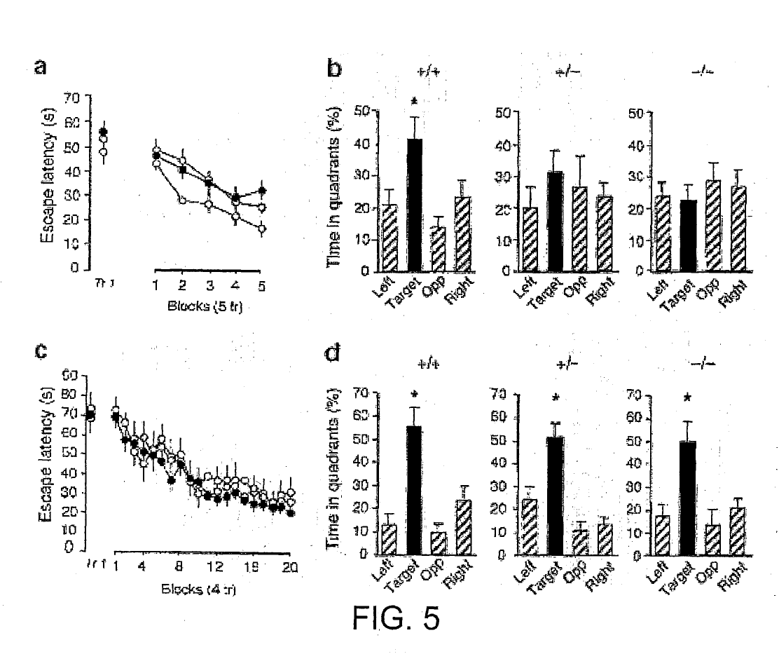

In the first task we tested spatial learning and memory in an open field water maze, using a massed training protocol where the acquisition phase was conducted within a two-hour period. All genotypes took the same length of time to find the escape platform in the first training trial (FIG. 5a). As the training progressed there was a reduction in escape latencies of all three genotypes. However, both +/− and −/− mice took significantly longer than the +/+mice, suggesting a learning deficit (F(2,24)=8.14, P<0.01). A corresponding memory deficit was evident in a probe trial performed 48 hours after training, during which +/+ mice showed a significant preference for the target quadrant (F(3,32)=6.01, P<0.01) whereas +/− and −/− (F<1 for both genotypes) mice showed no spatial bias for the training quadrant (FIG. 5b). There was no difference between the genotypes in swim speeds and mice did not exhibit floating behaviour: the deficit observed in the acquisition, and retention (and/or recall) of this, task, could not therefore be explained by aberrant motor behaviour.

Tasks requiring a higher level of learning can often be better achieved with extended and distributed training (Rasmussen, et al., (1989) Psychobiol. 17, 335-348). Using a protocol in which training was distributed and extended over 10 days rather than one day, all genotypes showed similar learning curves during the acquisition phase (FIG. 5c). A long-term retention test showed that all groups spent more time in the training quadrant in a probe trial given 8 days later (all P values<0.01; FIG. 5d). Thus, with extended training zif268 mutant mice show normal acquisition and long-term recall.

As spatial learning is a complex associative task in which learning takes place over several trials, we examined performance in three further tasks in which learning is achieved after a single paired trial (conditioned taste aversion and social transmission of food preference) or a brief training period (novel object recognition). In this way we hoped to define a time point-dependent requirement for zif268 activity by measuring the strength of associations at different times after pairing. In a non-spatial—task, conditioned taste aversion, water-deprived mice learn to associate a novel taste (15% sucrose solution) with the malaise induced by injection of lithium chloride (Garcia, et al., (1955) Science 122, 157-158). When subsequently offered the choice between water and sucrose solution, animals tend to avoid the sucrose solution. This task is not hippocampal-dependent, but requires structures including the basolateral amygdala and the insular cortex. Moreover, zif268 mRNA is upregulated following induction of LTP in the pathway connecting these two structures (Jones, et al., (1999) J. Neurosci. 19, RC36). We found, 24 h after LiCl injections, that +/+ mice showed significant aversion to the sucrose (P<0.05 vs. NaCl-injected +/+ mice FIG. 6). Neither +/− nor −/− mice exhibited significant aversion at 24 h (P>0.1) suggesting they were unable to maintain an association between the sucrose and malaise for this length of time. Analysis of variance confirmed a significant difference in aversion index between genotypes (F(2,24) 9.58; P<0.01 for three LiCl-injected groups). The same effects were also observed 48 h after LiCl injections (data not shown). The three groups that were injected with NaCl showed no preference for sucrose or water, either 24 or 48 h after injection.

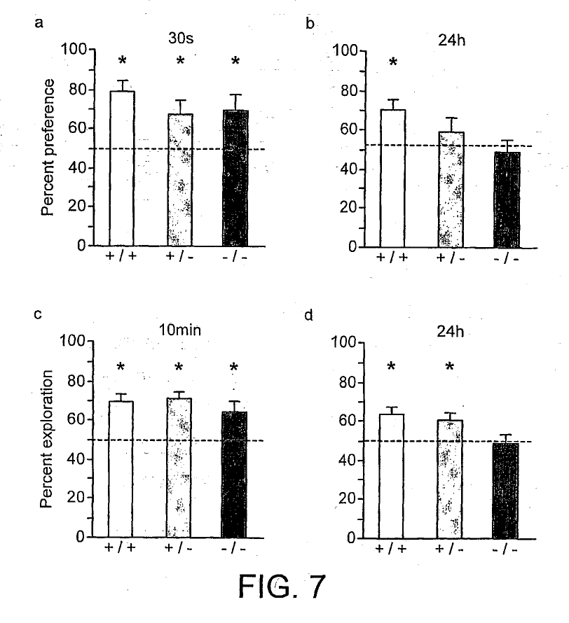

These data show that zif268 is necessary for the formation of long-term memory for an association formed in a single trial. Because of the malaise induced by injections of LiCl, we were unable to test the mice at short intervals, so it was not possible to determine whether or not short-term memory for the association was affected in zif268 mutants. Therefore, in two further experiments we used a within animal protocol to test for both short and long-term memory. In the first task, we used social transmission of food preference to test one-trial learning both immediately after learning and 24 h later. This is an olfactory discrimination task in which rodents show preference for a novel food that has recently been smelled on the breath of another (demonstrator) animal (Strupp, B. J. & Levitsky, D. A. (1984) J. Comp. Physiol. 98, 257-266). Food-deprived demonstrator mice were given either cocoa or coriander scented food to eat over a period of two hours and then allowed to interact with observer mice for 20 min. Food-deprived observer mice were then allowed free choice between cocoa and coriander either 30 s after the interaction or 24 h later. Thirty seconds after the interaction, all genotypes showed a significant preference for the food they had smelled on the breath of the demonstrators (all P values<0.05). Twenty-four hours later, however, while +/+ mice still showed a preference for the demonstrated food, +/− and −/− mice consumed similar amounts of both food types (see FIG. 7a). Analysis of the consumption of each of the scented foods (F(1,53)=1.05; P>0.05) and the amount of food consumed by the demonstrators (F(2,52)=1.95; P>0.05) showed no difference between genotypes, suggesting that there was neither a bias towards a particular food, nor that the demonstrators in one genotype failed to consume enough food to transmit the smell. These data show that zif268 is important for the formation of long-term memory for an olfactory event, while short-term memory for the same event is not dependent on zif268.

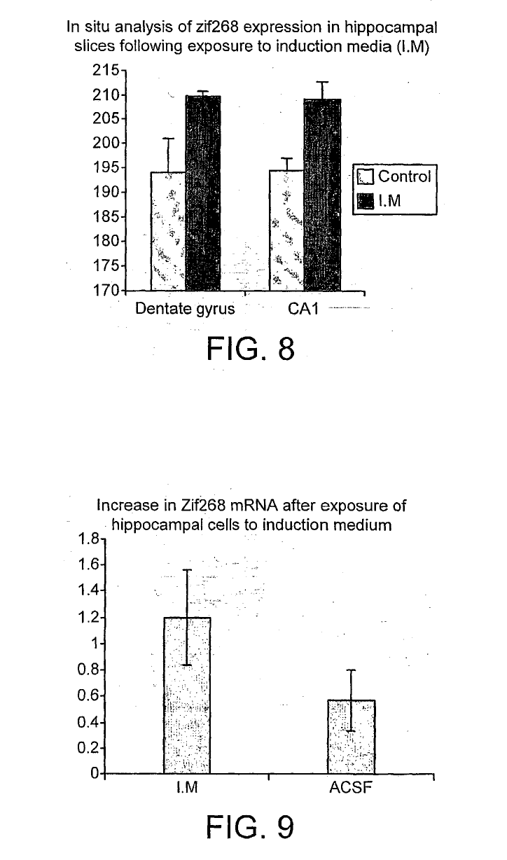

Rodents tend to-explore a novel object in preference to a familiar object. Novel object recognition is hippocampal-dependent (Rampon, C. et al. (2000) Nature Neurosci 3, 238-244) and we used this as a second task in which to assess both-short and long-term memory in the same groups of animals. Mice were allowed to explore two objects for a total of 20 min. Following a 10 min or-24 h delay, one of the familiar objects was replaced with a novel object and the time spent exploring the novel and familiar objects was measured. At the 10 min delay, mice of all genotypes spent significantly longer exploring the novel object than the familiar object (all P values<0.05; FIG. 7b), indicating similar levels of motivation and short-term memory. In contrast, when a 24 h delay was imposed between exploring the familiar and novel objects, +/+ and +/− mice still preferentially explored the novel object (P<0.01) whereas −/− mice did not (P>0.05; FIG. 8b). Thus, as with social transmission of food preference, +/+ mice were able to retain information acquired 24 h earlier whereas −/− mice could not. The performance of +/− mice was again intermediate although, in contrast to their performance in social transmission of food preference, they exhibited significant retention at 24 h.

These results demonstrate an absence of late phase LTP in the dentate gyrus of freely moving mice with a targeted inactivation of the immediate early gene zif268. The observation that LacZ reporter mRNA is upregulated after tetanic stimulation suggests that signalling events upstream of zif268 transcription are not affected in mice containing the mutant gene. We conclude that the absence of late LTP is due to a failure in the synthesis of downstream effector proteins encoded by genes for which zif268 is an obligatory transcription factor.

As a consequence of pituitary and ovarian defects, homozygous zif268 mutant mice have a reduced body size and are sterile, although heterozygous mice are phenotypically normal in terms of size and fertility. However, LTP decayed at a similar rate in both, heterozygous and homozygous mice, which also showed similar deficits in two of the learning tasks that placed demands on long-term memory. This dissociation between endocrine abnormalities and electrophysiological and behavioural phenotypes suggests that endocrine dysfunction does not contribute to the observed impairments in synaptic plasticity or memory. Histological examination of the hippocampus using cellular, neuronal and presynaptic markers confirmed similar cell densities and hippocampal architecture in wildtype and mutant mice, showing that disruption of zif268 had no gross effects on anatomical circuitry.

We found basal synaptic transmission, neuronal excitability, and short-term plasticity were normal in mutant mice. All genotypes showed an equivalent magnitude of LTP for the first hour following its induction. However, whereas LTP was maintained for at least 48 h after induction in wildtype mice, LTP in both heterozygous and homozygous mutant mice decayed to baseline within 24 h. The level of zif68 mRNA in heterozygous animals is approximately half that seen in wildtype mice (data not shown), suggesting that this is insufficient to achieve the levels of zif268 activation required for the successful expression of late LTP.

Other immediate early genes have also been implicated in both LTP and learning. For example, mRNA for Homer, a protein that binds metabotropic glutamate receptors, is upregulated following LTP induction (Brakeman, P. R. et al. (1997) Nature 386: 284-288; Kato, A. et al. (1998) J. Biol. Chem. 273: 23969-23975). It has recently been shown, using antisense techniques, that Arc (also known as Arg 3.1), another mRNA species upregulated after LTP (Link, W. et al. (1995) Proc. Natl. Acad. Sci. USA 92: 5734-5738; Lyford, G. L. et al. (1995) Neuron 14, 433-445), is necessary for both late LTP and spatial learning (Guzowski, J. F. et al. (2000) J. Neurosci. 20: 3993-4001). Together with our results, these data suggest that several immediate early genes act together to establish late LTP.

To examine the role of Zif268 in short- and long-term memory, we used a variety of behavioural tasks making, use of single or repeated training, different types of reinforcement, and the processing of spatial or non-spatial information. Some of these tasks were hippocampal dependent, and some were not. Our results provide evidence that at least some forms of short-term memory are intact in the zif268 mutant mice since they demonstrated normal levels of spontaneous alternation, an innate behaviour which relies upon spatial working memory. In common with early LTP, spontaneous alternation requires activation of the NMDA receptor and phosphorylation of protein kinase C (Walker, D. L. & Gold, P. E. (1994) Behav. Neural Biol. 62, 151-162), events that are upstream of gene transcription, and appear to be sufficient to mediate short-term memory. zif268 mutant mice were also able to perform olfactory discrimination in social transmission of food preference and visual discrimination in an object recognition task, provided no delay was imposed. These results are consistent with the notion that loss of functional zif268 does not affect short-term memory processes.

In contrast to the lack of effect on short-term memory, long-term memory in zif68 mutant mice was severely impaired. In three forms of learning, conditioned taste aversion, olfactory discrimination, and novel object recognition, homozygous mice exhibited no significant recall when tested 24 h later. Interestingly, whereas the heterozygous mice showed a similar deficit in learning as the homozygous mice in olfactory discrimination and the conditioned taste aversion, they showed no marked impairment in the object recognition task. Nevertheless, these findings provide support for the notion that zif268 plays a critical role in the consolidation or stabilisation of the memory trace. Spatial navigation is a more complex type of learning, requiring several trials to reach criterion. During massed training, both heterozygous and homozygous mice showed reduced performance and a severe deficit in long-term spatial memory assessed in probe trials 24 h later, suggesting that zif268 is required for memory consolidation during and following the process of learning. We found that spatial memory deficits in the zif268 mutant mice could be completely rescued by extended and distributed training. Similar deficits in the consolidation of learning and the potential for rescue by extended training have been reported in mice with targeted disruption of the gene encoding the cAMP response element binding protein CREB (Kogan, J. H. et al. (1996) Current Biology 7, 1-11). In addition, genetic studies in Drosophila have suggested that the ability to form long-term memories following disruption of CREB signalling is influenced by the temporal structure of the training schedule (Yin, J., et al. (1995) Cell 81, 107-115). The upstream regulatory elements, of the zif268 gene include six serum response elements (SRE) and two cAMP response elements (CRE), and LTP-dependent transcriptional regulation of zif268 is controlled by the mitogen-activated protein kinase (MAPK) pathway. Both CREB and MAPK signalling have been implicated in synaptic plasticity and certain types of learning. Our data, therefore, strengthen the evidence that zif268 is an essential participant in the signalling cascade required for synaptic and behavioural plasticity.

The results described here-establish an isomorphism between hippocampal LTP and learning with respect to the effects of the zif268 mutation: short-term plasticity and short-term memory are unaffected whereas long-term plasticity and, long-term memory are impaired. zif268 may also be important for synaptic plasticity in other brain structures, such as the insular cortex, a region which displays plasticity-related zif268 activation (Jones, et al., (1999) J. Neurosci. 19, RC36), and which mediates conditioned taste aversion, a task which we have also shown to be impaired in zif268 mutant mice.

In summary, the data presented here (Jones, M. W., et al., (2001) Nature Neurosci. 4: 289-296) establish that activation of the immediate early gene zif268 is essential for stabilising synaptic plasticity in the hippocampus and for the consolidation of spatial and non-spatial forms of long-term memory. This supports the suitability of zif268 expression as a reporter for use in this invention.

### Example 2

Chemical Induction of LTP is Accompanied by Zif268 Expression

In order to assess the relationship between chemical LTP induction and zif268 expression, native zif268 expression was analysed in a number of experimental systems which model in vivo LTP induction.

zif268 Expression in Hippocampal Slices

In situ hybridisation was performed against sections cut from hippocampal slices that had been exposed in vitro to the LTP-induction medium (or control ACSF) for 10 min then fixed 30 min later before sectioning and hybridising with radioactive zif268 antisense probe. The induction medium: consists of: NaHCO₃, 24 mM; glucose, 10 mM; KH₂PO₄, 1.25 mM; MgCl₂, 0.1 mM; KCl, 5 mM; NaCl, 95 mM; CaCl₂, 5 mM; tetraethylammonium 25 mM, with pH adjusted to 7.4 Film overlays, after suitable exposure time, were quantified by densitometric scanning of the indicated hippocampal region. Zif268 mRNA is elevated by induction medium in dentate gyrus, as after the more-usual electrical stimulation protocols (FIG. 8). This experiment is one of several validating the chemical induction procedure by comparison with ‘normal’ LTP.

Northern Blot Analysis

Northern blot analysis of total mRNA from ‘cultured cells shows’ that hippocampal neurons in dissociated cell culture, just as in organised brain slices, sustain elevated zif268 expression (normalised against levels of actin mRNA, which are not expected to change) 30 min after 10 min exposure to the chemical LTP-induction medium (FIG. 9). This result validates the use of dissociated cells in LTP assays.

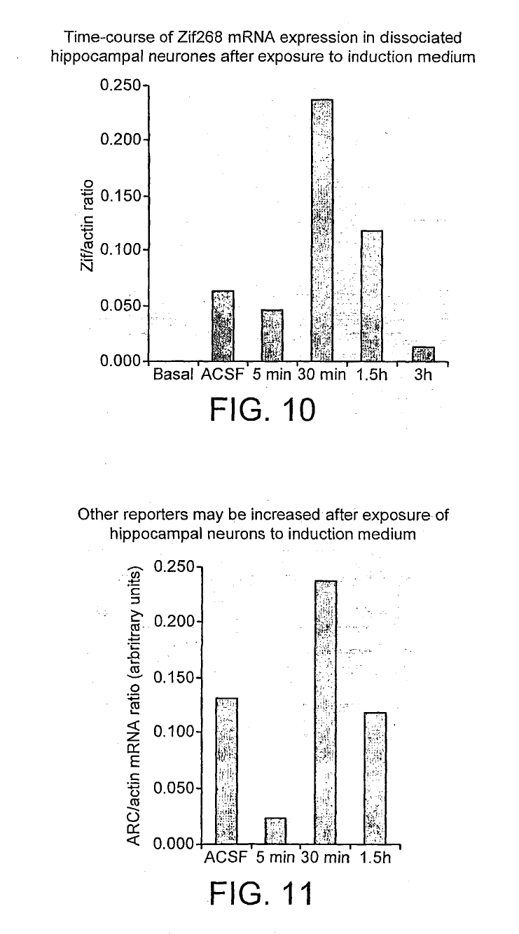

FIG. 10 shows the results of an experiment similar to that shown in FIG. 10, but with varying intervals between induction medium exposure and subsequent cell lysis and mRNA extraction. The peak in zif268 expression after 30 min established the optimal delay for mRNA-based assays. Similar optimisation of protein-based immunohistochemical assays indicates an optimum 2 h post-exposure delay.

FIG. 11 shows an experiment similar to FIG. 10, except the Northern blots have been probed for Arc mRNA. The data show that expression of Arc, like zif268, is induced by 10 min exposure to induction medium, with mRNA levels peaking at 30 minutes post-exposure. Thus Arc as well as zif268 should be suitable for use as a reporter in this invention.

### Example 3

Chemical Induction of LTP is Detected by Zif268 Immunocytochemical Assay

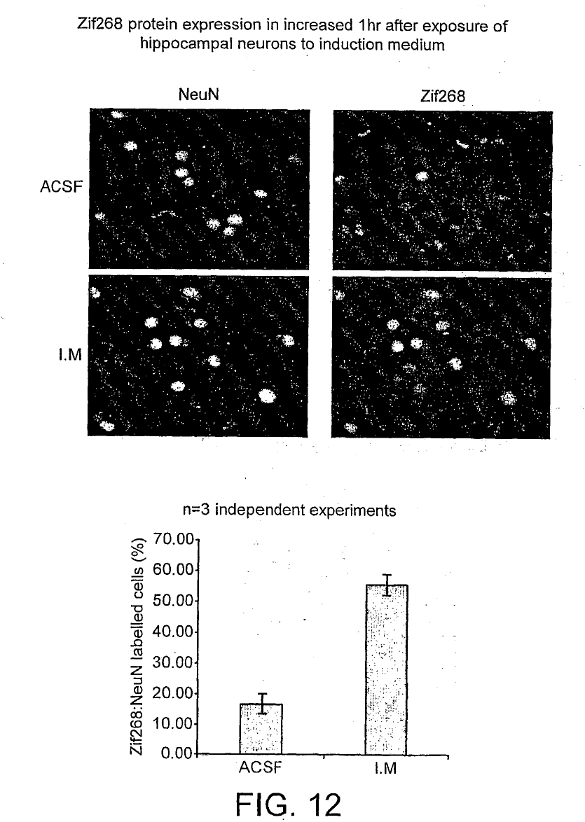

Hippocampal neurons were prepared from late embryonic or 1^{st} postnatal day Sprague-Dawley rates, and grown in dissociated cell culture in microtitre plates. After 7-21 days in vitro, serum-containing medium was replaced with serum-free medium for 24 h. This medium was then replaced with ACSF or with induction medium for 10 minutes; these solutions were then replaced with serum-free medium. After 1 hour, cells were fixed by replacement of serum-free medium with phosphate-buffered saline containing 4% paraformaldehyde. Immunolabelling was then carried out with rabbit antiserum against zif268 and mouse antibodies against the neuron-specific antigen NeuN. After washing, bound antibodies were visualised by incubation with Alexa488-labelled anti-rabbit IgG and Cy3-labelled anti-mouse IgG (FIG. 12 top). LTP induction led to an approximately; four-fold increase in the incidence of double-labelled cells (i.e., zif268-expressing neurons; FIG. 12, bottom).

### Example 4

Chemical Induction of LTP is Detected by Zif268/β-Galactosidase Assay

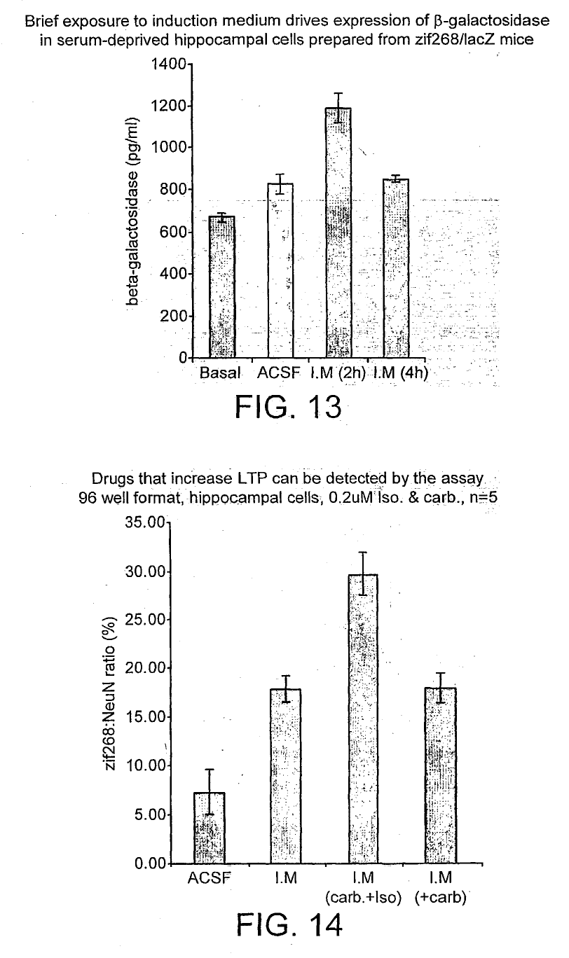

Dissociated hippocampal neuronal cultures were prepared from embryonic wild-type and embryonic −/− and +/− zif268 knockout mice of the line used in Example 1. As noted above, the mutation in these animals involved the insertion of a lacZ-neo cassette containing a polyadenylation site between the promoter and its coding sequence, which caused the lacZ gene to be transcribed in place of the zif268 gene. Cells were treated as in Example 3, but 2 or 4 hours after exposure to induction medium or ACSF control, cells were solubilized, and levels of β-Galactosidase were determined by incubation with the chromogenic β-Galactosidase substrate, X-gal, according to standard methods, and expressed in terms of total protein (FIG. 13). As above, LTP induction was detected as a significant increase in β-Galactosidase 2 h after exposure to the induction medium.

### Example 5

Drugs that Increase or Decrease LTP can be Detected by the Assay

An immunocytochemical assay was carried out as in Example 3, with cells exposed to ACSF, to induction medium, to induction medium with the addition of carbachol and isoproterenol (0.2 μM each) or to induction medium with the addition of carbachol only (0.2 μM). Carbachol alone had no effect, but the combination of carbachol and isoproterenol significantly increased neuronal zif268 expression (FIG. 14), a result similar to that obtained with electrically-induced LTP in acutely-prepared hippocampal slices (Watabe, A. M., et al., (2000) J. Neurosci. 20: 5924-31).

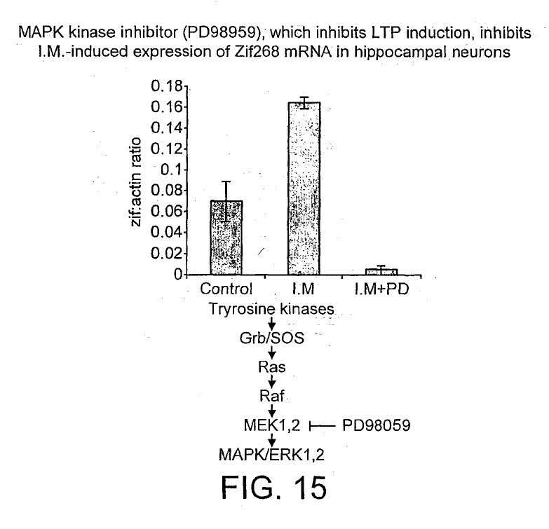

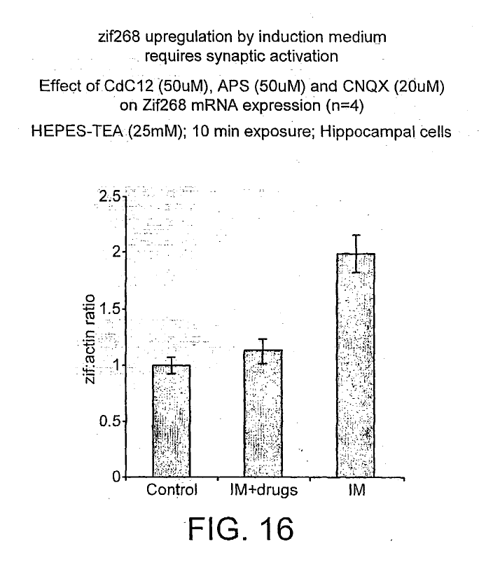

Zif268 mRNA assays were carried out as in Example 2, with cells exposed to ACSF control, to induction medium, or to induction medium with the addition of PD98959 (an inhibitor of MAPK kinase) (FIG. 15) or with the addition of a mixture of the calcium channel blocker cadmium and the glutamate receptor antagonists AP5 and CNQX (FIG. 16). Both additions to the induction medium significantly reduced the induction of zif268 mRNA expression, a result similar to the effects of these added drugs on electrically-induced LTP in acutely-prepared hippocampal slices.

### Example 6

Luciferase Assay Using Zif68 Regulatory Sequence

All media and reagents used for routine cell culture are purchased from Gibco (Grand Island, N.Y.), Hazelton (Lenexa, Kans.), or Whittaker M. A. Biologicals (Walkersville, Md.). Fetal calf serum CFCS) is from Hyclone (Logan, Utah).

A human neural carcinoma cell line is used for the transfection of plasmids carrying the promoter and regulatory elements of the human zif268 gene. These cells are maintained in DMEM supplemented with 10% FCS.

Unless otherwise indicated, molecular cloning procedures are performed essentially according to Sambrook et al. Oligonucleotides are synthesized by the beta-cyanoethyl phosphoramidite method according to protocols provided by the manufacturer of the DNA-synthesizer (Model 380A, Applied Biosystems (Foster City, Calif.).

A mammalian expression shuttle vector is designed to allow the construction of the promoter-reporter gene fusions to, be used in high-throughput screens to identify transcriptionally modulating chemicals.

The firefly luciferase gene is removed from the plant expression plasmid pD6432 (Ow, D. W., et al., (1986), Science 234:856-859) as a 1.9 kb BamHI fragment and cloned into the BamHI site of pSVL (Pharmacia, Piscataway, N.J.), a mammalian expression vector containing the SV40 promoter. The resulting plasmid is digested with XhoI and SalI to produce a 2.4 kb fragment: containing the luciferase coding sequences and the SV40 late polyadenylation site. This fragment is inserted into the XhoI site of a eukaryotic expression vector containing the zif268 promoter (Pharmacia, Piscataway, N.J.). The resulting zif268 promoter-luciferase fusion plasmid (pLuc-Z) is used to transfect neural cells as described below. Similar constructs can be made using luciferase vectors from Clontech (Palo Alto, Calif.).

A 476 b fragment containing a dimeric SV40 polyadenylation site is then cloned into the BclI site of pLuc-Z. To do this, a 238 bp BclI(BamHI fragment is obtained from SV40 genomic DNA. (BRL), ligated, digested with BclI/BamHI, gel isolated, and inserted into pLuc-Z, resulting in the vector pLuc-Z2.

The neomycin resistance gene (neo) is then placed under control of the Herpes Simplex Virus thymidine kinase (HSV-TK) promoter to generate a resistance cassette which is free of known enhancer sequences. To do this the HSV-TK promoter is synthesized using four oligonucleotides designed according to published sequence information (McKnight, S. L. (1982), Cell, 31:355), and including an SfiI restriction site 5′ of the HSV-TK sequences. These oligonucleotides are phosphorylated, annealed, ligated and inserted into pLuc-Z digested previously with HindIII NheI, generating the vector pLuc-Z3.

The vector pLuc-Z3-is transformed into neural cell line cells by electroporation and its presence is selected for according to G418 resistance.

Liquid Scintillation Counter Bioluminescence Assay

To assay for luciferase expression in transient expression assays in the transfected cell lines, cells are incubated with the LTP inducer forskolin in serum free defined medium, washed 3 times with Dulbecco's phosphate-buffered saline (D-PBS, Gibco) and lysed in Lysis Buffer 1 (50 mM Tris acetate pH 7.9, 1-mM EDTA, 10 mM magnesium acetate, 1 mg/ml bovine serum albumin [BSA], 0.5% Brij 58, 2 mM ATP, 100 mM dithiothreitol [DTT]). All reagents are obtained from Sigma except for DTT which is from Boehringer Mannheim. After lysis debris sedimented by brief centrifigation, and 950 μl of supernatant extract are added to a glass scintillation vial. Samples are counted individually in an LKB (Gaithersburg, Md.) scintillation counter on a setting which allows measurement of individual photons by switching off the coincidence circuit. The reaction is started by addition of 50 μl of 2 mM luciferin (Sigma, St; Louis, Mo. or Boehringer Mannheim, Indianapolis, Ind.) in Buffer B (Buffer B-Lysis Buffer 1 without Brij 58, ATP and DTT) to the 950 μl of lysate. Measurement is started 20 seconds after luciferin addition and continued for 1 minute. Results are normalized to protein concentration using the Bradford protein assay (BioRad, Richmond Calif.) or to cell numbers using Trypan Blue (Sigma) exclusion counting in a hemocytometer.

Expression of luciferase from the zif268 promoter, as judged by scintillation counting, will increase in response to forskolin addition as a result of zif268 promoter stimulation.

### Example 7

High Throughput Screening

Cell plating: Dynatech Microlite 96 well plates are pretreated for cell attachment by Dynatech Laboratories, Inc.(Chantilly, Via). Alternatively, the 96 well plates are treated with 50 μl per well of human fibronectin (hFN, 15 μg/ml in PBS, Collaborative Research, Bedford, Mass.) overnight at 37° C. hFN-treated plates are washed with PBS using an Ultrawash 2 Microplate Washer (Mynatech Labs), to remove excess hFN prior to cell plating. Human cortical neuronal cell lines HCN-1A and HCN-2, grown according to their supplier's directions (American Type Culture Collection) are washed with PBS, harvested by trypsinization, and counted using a hemocytometer and the Trypan Blue exclusion method according to protocols provided by Sigma, St. Louis, Mo. Chemical Company. Cells are then diluted into serum free defined media (with 0.2 mg/ml G418), and 0.2 ml of cell suspension per well is plated onto Dynatech treated plates or hFN-treated plates using a Cetus Pro/Pette (Cetus, Emeryville Calif.). Plates are incubated overnight at 37° C. in a humidified 5%-CO₂-atmosphere, and then differentiated by further growth for 7 days in the presence f IBM, cyclic AMP and NGF. They are then transferred to serum-free medium for 24-48 hours before addition of test compounds.

Addition of Chemicals to Cells: Chemicals are dissolved in DMSO at concentrations of 3-30 mg/ml. A liquid handling laboratory work station (RSP 5052, Tecan U.S. Chapel Hill, N.C.) is used to dilute the chemicals (three dilutions; 5 fold, 110 fold, and 726 fold). 10 μl of each dilution are added to each of quadruplicate samples of cells contained in the wells of 96-well Dynatech Microlite Plates. Cell plates are then shaken on a microplate shaker (Dynatech, medium setting, 30 sec.) and incubated for 6 hours at 37° C., 5% CO₂.

Bioluminescence Assay: After incubation with OSI-file chemicals, cell plates are washed 3 times with PBS using an Ultrawash 2 Microplate Washer (Dynatech Labs) and 75 μl of Lysis Buffer 2 are added to each well (Lysis Buffer 2 is the same as Lysis buffer 1 except that the ATP and DTT concentrations are changed to 2.67 mM and 133 mM, respectively). Bioluminescence is initiated by the addition of 25 μl 0.4 μM Luciferin in Buffer B to each well, and is measured in a Dynatech ML 1000 luminometer following a 1 minute incubation at room temperature. Data are captured and analyzed using Lotus-Measure (Lotus) software.

Alternatively a fully automated device as described in U.S. patent application Ser. No. 382,483 is used to incubate luciferase reporter cells in 96-well microtiter plates, transfer chemicals and known transcriptional modulators to the cells, incubate cells with the chemicals, remove the chemicals by washing with PBS, add lysis buffers the cells and measure the bioluminescence produced.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are apparent to those skilled in molecular biology or related fields are intended to be within the scope of the following claims.

1. A method for determining whether one or more compounds is a potential modulator of long-term potentiation (LTP) in the brain, comprising the steps of:
a) providing a cell which expresses a gene under the control of a regulatory sequence naturally associated with a gene whose expression is associated with LTP;
b) contacting the cell with one or more compounds;
c) determining the ability of the compound or compounds to modulate the expression of the gene; and
d) correlating the modulation of gene expression with the ability to modulate LTP in the brain.

2. A method according to claim 1, wherein the cell is a mammalian cell.

3. A method according to claim 1, wherein the gene is endogenous to the cell.

4. A method according to claim 1, wherein the gene is detected by a fluoroimmunoassay.

5. A method according to claim 1, wherein the gene is a reporter gene.

6. A method according to claim 1, wherein the gene whose expression is associated, with LTP is selected from the group consisting of zif268, arc, Egr3, CRE c-fos, fra-1, fra-2, fosB, cjun, junB, jund, C/EPB, CaMKII, PKC, PKA, ERK-2, Raf-B, BAD-2, homer, frequenin, AKAP150, BDNF, and NMDA-, AMPA- and metabotropic-glutamate receptors.

7. A method according to claim 5, wherein the reporter gene is selected from the group consisting of genes which encode luminescent proteins, fluorescent proteins and enzymes capable of catalysing a reaction which has a detectable result.

8. A method according to claim 7, wherein the reporter gene encodes luciferase, green fluorescent protein, β-lactamase and β-galactosidase.

9. A method according to claim 1, wherein expression of the gene is detectable optically.

10. A method according to claim 1, wherein the transformed cell is comprised in a transgenic organism.
